(19) United States
(12) Patent Application Publication
(10) Pub. No.: US 2012/0203110 A1
(43) Pub. Date: Aug. 9, 2012

(54) ULTRASOUND DIAGNOSTIC APPARATUS AND ULTRASOUND IMAGE PRODUCING METHOD

(75) Inventor: Katsuya YAMAMOTO, Kanagawa (JP)

(73) Assignee: FUJIFILM CORPORATION, Tokyo (JP)

(21) Appl. No.: 13/362,544

(22) Filed: Jan. 31, 2012

(30) Foreign Application Priority Data
Feb. 9, 2011 (JP) 2011-026076

(51) Int. Cl. A61B 8/14

(52) U.S. Cl. 600/447

(57) ABSTRACT

An ultrasound diagnostic apparatus includes a channel selector for selecting channels that are simultaneously available for reception from a plurality of channels of a transducer array, and a controller for controlling a transmission driver to sequentially transmit a plurality of ultrasonic beams having different frequencies corresponding to a plurality of measuring depth regions from the transducer array, controlling reception signal processors and an image producer to form a same frame by receiving ultrasonic echoes having a frequency corresponding to each of the plurality of measuring depth regions, and controlling the channel selector to increasingly reduce the number of channels simultaneously available for reception as a measuring depth in the plurality of measuring depth regions decreases.

### BACKGROUND OF THE INVENTION

The present invention relates to an ultrasound diagnostic apparatus and an ultrasound image producing method and particularly to reduction of the amount of heat generated in an ultrasound probe of an ultrasound diagnostic apparatus for giving a diagnosis based on an ultrasound image produced by transmission and reception of ultrasonic waves from a transducer array of the ultrasound probe.

Conventionally, ultrasound diagnostic apparatus using ultrasound images are employed in medicine. In general, this type of ultrasound diagnostic apparatus comprises an ultrasound probe having a built-in transducer array and an apparatus body connected to the ultrasound probe. The ultrasound probe transmits ultrasonic waves toward a subject and receives ultrasonic echoes from the subject, and the apparatus body electrically processes the reception signals to generate an ultrasound image.

With such ultrasound diagnostic apparatus, heat is generated in the transducer array as it transmits ultrasonic waves.

The ultrasound probe is often encased in a housing of a size that can be readily held by an operator in a single hand because normally a diagnosis is given as the operator places the ultrasound transmission/reception surface of the transducer array in contact with a surface of a subject's body by holding the ultrasound probe in a single hand. Therefore, the heat generated in the transducer array may raise the temperature inside the housing of the ultrasound probe.

In recent years, there has been proposed an ultrasound diagnostic apparatus having an ultrasound probe with a built-in circuit board for signal processing for effecting digital processing of reception signals outputted from the transducer array before transmitting the reception signals to the apparatus body via wireless or wired communication in order to reduce the effects of noise and obtain a high-quality ultrasound image.

The ultrasound probe that effects digital processing of this kind is subject to generation of heat in the circuit board also during processing of the reception signals, and therefore the temperature rise in the housing needs to be suppressed to assure stable operations of the circuits on the board.

As for a countermeasure against the temperature rise in the ultrasound probe, reference is made to JP 2005-253776 A describing an ultrasound diagnostic apparatus wherein the conditions for driving the transducer array are automatically changed according to the temperature of the surface of the ultrasound probe. The temperature of the surface of the ultrasound probe is kept at an appropriate temperature by reducing, for example, driving voltages for transducers, a number of channels for transmission, a repetition frequency of a transmission pulse, and a frame rate as the surface temperature increases.

### SUMMARY OF THE INVENTION

However, the apparatus described in JP 2005-253776 A where the conditions for driving the transducer array for transmission are changed cannot cope with the heat generated by the reception process in the ultrasound probe performing the above digital processing.

An object of the present invention is to eliminate the above problems associated with the prior art and provide an ultrasound diagnostic apparatus and an ultrasound image producing method enabling acquisition of a high-quality ultrasound image while suppressing the temperature rise inside the ultrasound probe.

An ultrasound diagnostic apparatus according to the present invention comprises:

an ultrasound probe including a transducer array with a plurality of channels;

a transmission driver for transmitting an ultrasonic beam from the transducer array toward a subject;

reception signal processors for processing reception signals outputted from the transducer array having received ultrasonic echoes from the subject;

an image producer for producing an ultrasound image based on the reception signals processed by the reception signal processors;

a channel selector for selecting channels that are simultaneously available for reception from the plurality of channels; and

a controller for controlling the transmission driver to sequentially transmit a plurality of ultrasonic beams having different frequencies corresponding to a plurality of measuring depth regions from the transducer array, controlling the reception signal processors and the image producer to form a same frame by receiving ultrasonic echoes having a frequency corresponding to each of the plurality of measuring depth regions, and controlling the channel selector to increasingly reduce the number of channels simultaneously available for reception as a measuring depth in the plurality of measuring depth regions decreases.

An ultrasound image producing method according to the present invention comprises the steps of:

sequentially transmitting a plurality of ultrasonic beams having different frequencies corresponding to a plurality of measuring depth regions from a transducer array in an ultrasound probe;

increasingly reducing the number of channels simultaneously available for reception as a measuring depth in the plurality of measuring depth regions decreases; and

forming a same frame by receiving ultrasonic echoes having a frequency corresponding to each of the plurality of measuring depth regions to produce an ultrasound image.

### BRIEF DESCRIPTION OF DRAWINGS

FIG. 1 is a block diagram illustrating a configuration of an ultrasound diagnostic apparatus according to Embodiment 1 of the invention.

FIG. 2 illustrates how an imaging region is divided in Embodiment 1.

FIG. 3 is a timing chart illustrating an operation of transmittance and reception of ultrasonic waves in Embodiment 1.

FIGS. 4A to 4C illustrate how a frame correlation processing is performed in Embodiment 1, FIG. 4A being a timing chart in a first frame, FIG. 4B being a timing chart in a second frame, FIG. 4C being a timing chart in a third frame.

FIG. 5 is a block diagram illustrating a configuration of an ultrasound probe in Embodiment 2.

FIG. 6 is a graph illustrating a temporal variation in temperature inside the ultrasound probe and temperature thresholds in Embodiment 2.

FIG. 7 illustrates how an imaging region is divided depending on temperature ranges in Embodiment 2.

FIG. 8 illustrates how an imaging region is divided in Embodiment 3.

### DETAILED DESCRIPTION OF THE INVENTION

Embodiments of the present invention will now be described below based on the appended drawings.

### Embodiment 1

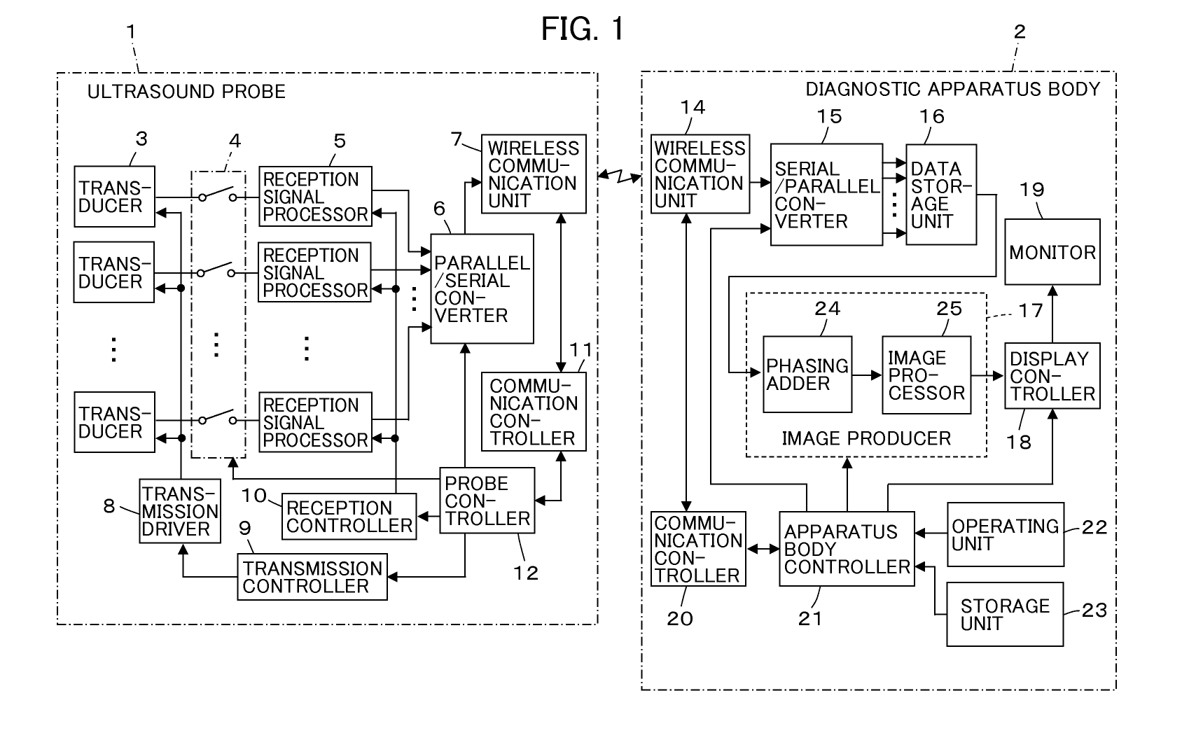

FIG. 1 illustrates a configuration of the ultrasound diagnostic apparatus according to Embodiment 1 of the invention. The ultrasound diagnostic apparatus comprises an ultrasound probe 1 and a diagnostic apparatus body 2 that is connected to the ultrasound probe 1 via wireless communication.

The ultrasound probe 1 comprises a plurality of ultrasound transducers 3 constituting a plurality of channels of a unidimensional or two-dimensional transducer array, and reception signal processors 5 are connected to the transducers 3 via a channel selector 4, and a wireless communication unit 7 is connected to the reception signal processors 5 via a parallel/serial converter 6. A transmission controller 9 is connected to the transducers 3 via a transmission driver 8, a reception controller 10 is connected to the reception signal processors 5, and a communication controller 11 is connected to the wireless communication unit 7. A probe controller 12 is connected to the channel selector 4, the parallel/serial converter 6, the transmission controller 9, the reception controller 10, and the communication controller 11.

The transducers 3 each transmit ultrasonic waves according to driving signals supplied from the transmission driver 8 and receive ultrasonic echoes from a subject to output reception signals. Each of the transducers 3 is composed of a vibrator comprising a piezoelectric body and electrodes each provided on both ends of the piezoelectric body. The piezoelectric body may be composed, for example, of a piezoelectric ceramic typified by a PZT (titanate zirconate lead), a polymeric piezoelectric device typified by PVDF (polyvinylidene flouride), and a monocrystal typified by PMN-PT (lead magnesium niobate lead titanate solid solution).

When the electrodes of each of the vibrators are supplied with a pulsed voltage or a continuous-wave voltage, the piezoelectric body expands and contracts to cause the vibrator to produce pulsed or continuous ultrasonic waves. These ultrasonic waves are combined to form an ultrasonic beam. Upon reception of propagating ultrasonic waves, each vibrator expands and contracts to produce an electric signal, which is then outputted as an reception signal.

The transmission driver 8 includes, for example, a plurality of pulsers and adjusts the delay amounts of driving signals based on a transmission delay pattern selected by the transmission controller 9 so that the ultrasonic waves transmitted from the transducers 3 form a broad ultrasonic beam covering an area of a tissue inside the subject and supplies transducers 3 with adjusted driving signals.

The channel selector 4 comprises a plurality of switches connecting and disconnecting between the transducers 3 and the corresponding reception signal processors 5 and selects simultaneously available channels for reception among the channels of the transducer array according to an instruction from the probe controller 12 to connect the transducers 3 of the selected channels to the corresponding reception signal processors 5.

Under the control of the reception controller 10, each of the reception signal processors 5 allows the reception signal outputted from the corresponding transducer 3 to undergo quadrature detection or quadrature sampling process to produce a complex base band signal, samples the complex base band signals to generate sample data containing information on the area of the tissue, and supplies the parallel/serial converter 6 with the sample data. The reception signal processors 5 may generate sample data by performing high-efficiency coding data compression on the data obtained by sampling the complex baseband signals.

The parallel/serial converter 6 converts the parallel sample data generated by the reception signal processors 5 into serial sample data.

The wireless communication section 7 performs carrier modulation based on the serial sample data to generate transmission signals and supplies an antenna with the transmission signals so that the antenna transmits radio waves to transmit serial sample data. The modulation methods that may be employed herein include ASK (Amplitude Shift Keying), PSK (Phase Shift Keying), QPSK (Quadrature Phase Shift Keying), and 16 QAM (16 Quadrature Amplitude Modulation).

The wireless communication unit 7 transmits the sample data to the diagnostic apparatus body 2 through wireless communication with the diagnostic apparatus body 2, receives various control signals from the diagnostic apparatus body 2, and outputs the received control signals to the communication controller 11. The communication controller 11 controls the wireless communication unit 7 so that the sample data is transmitted with a transmission wave intensity that is set by the probe controller 12 and outputs various control signals received by the wireless communication unit 7 to the probe controller 12.

The probe controller 12 controls various components of the ultrasound probe 1 according to control signals transmitted from the diagnostic apparatus body 2.

The ultrasound probe 1 has a built-in battery, not shown, which supplies electric power to the circuits inside the ultrasound probe 1.

The ultrasound probe 1 may be an external type probe such as linear scan type, convex scan type, and sector scan type or a probe of, for example, a radial scan type used for an ultrasound endoscope.

On the other hand, the diagnostic apparatus body 2 comprises a wireless communication unit 14, which is connected to a data storage unit 16 via a serial/parallel converter 15. An image producer 17 is connected to the data storage unit 16, and a monitor 19 is connected to the image producer 17 via a display controller 18. A communication controller 20 is connected to the wireless communication unit 14, and an apparatus controller 21 is connected to the serial/parallel converter 15, the image producer 17, the display controller 18, and the communication controller 20. The apparatus controller 21 is connected to an operating unit 22 for an operator to perform input operations and to a storage unit 23 for storing operation programs.

The wireless communication unit 14 transmits various control signals to the ultrasound probe 1 through wireless communication with the ultrasound probe 1. The wireless communication section 14 demodulates the signal received by an antenna to output serial sample data.

The communication controller 20 controls the wireless communication unit 14 so that various control signals are transmitted with a transmission radio wave intensity that is set by the apparatus body controller 21.

The serial/parallel converter 15 converts the serial sample data outputted from the wireless communication unit 14 into parallel sample data. The data storage unit 16 is configured by a memory, a hard disk, or the like and stores at least one frame of sample data converted by the serial/parallel converter 15.

The image producer 17 performs reception focusing on each frame of sample data read out from the data storage unit 16 to generate an image signal representing an ultrasound diagnostic image. The image producer 17 includes a phasing adder 24 and an image processor 25.

The phasing adder 24 selects one reception delay pattern from a plurality of previously stored reception delay patterns according to the reception direction set by the apparatus controller 21 and, based on the selected reception delay pattern, provides the complex baseband signals represented by the sample data with respective delays and adds them up to perform the reception focusing. This reception focusing yields a baseband signal (sound ray signal) where the ultrasonic echoes are well focused.

The image processor 25 generates a B-mode image signal, which is tomographic image information on a tissue inside the subject, according to the sound ray signal generated by the phasing adder 24. The image processor 25 includes an STC (sensitivity time control) section and a DSC (digital scan converter). The STC section corrects the sound ray signal for the attenuation due to distance according to the depth of the reflection position of the ultrasonic waves. The DSC converts the sound ray signal corrected by the STC into an image signal compatible with the scanning method of an ordinary television signal (raster conversion), and generates a B mode image signal through required image processing such as contrast processing.

The display controller 18 causes the monitor 19 to display an ultrasound diagnostic image according to the image signals generated by the image producer 17. The monitor 19 includes a display device such as an LCD, for example, and displays an ultrasound diagnostic image under the control of the display controller 18.

While the serial/parallel converter 15, the image producer 17, the display controller 18, the communication controller 20, and the apparatus controller 21 in such diagnostic apparatus body 2 are each constituted by a CPU and an operation program for causing the CPU to perform various kinds of processing, they may be constituted by a digital circuit. The aforementioned operation program is stored in the storage unit 23. The recording medium in the storage unit 23 may be a flexible disk, MO, MT, RAM, CD-ROM, DVD-ROM or the like besides a built-in hard disk.

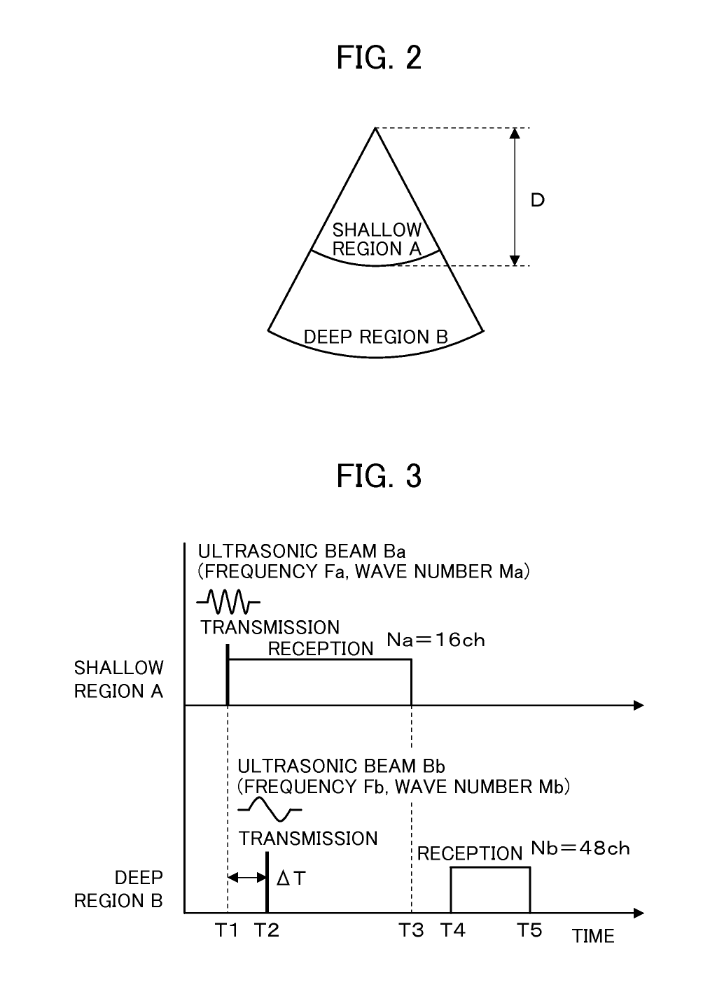

According to Embodiment 1, an imaging region is previously divided at a given depth D into two regions, a shallow region A and a deep region B, according to a measuring depth, as illustrated in FIG. 2. Two ultrasonic beams having different frequencies and different transmission timings from each other are transmitted for the shallow region A and the deep region B and each received by different numbers of channels simultaneously available for reception.

When the transducer array of the ultrasound probe has a total of 48 channels, for example, an ultrasonic beam Ba having a frequency or Fa and a wave number of Ma is transmitted while a number of channels simultaneously available for reception is set to Na=16 to measure the shallow region A, whereas an ultrasonic beam Bb having a frequency of Fb and a wave number of Mb is transmitted while a number of channels simultaneously available for reception is set to Nb=48 to measure the deep region B, as illustrated in FIG. 3.

The frequency Fb of the ultrasonic beam Bb for measuring the deep region B is set to a smaller value than the frequency Fa of the ultrasonic beam Ba for measuring the deep region A. The ultrasonic beam Bb for measuring the deep region B is transmitted at a timing delayed by a given time ΔT with respect to the ultrasonic beam Ba for measuring the shallow region A.

The above measuring conditions including the given depth D of the imaging region, the frequency Fa and the wave number Na of the ultrasonic beam Ba, the frequency Fb and the wave number Mb of the ultrasonic beam Bb, the numbers of channels, Na and Nb, simultaneously available for reception, and the given time ΔT may previously have been entered from the operating unit 22 of the diagnostic apparatus body 2 and stored in the storage unit 23.

The reception signal processors 5 of the ultrasound probe 1 therein comprises a high-pass filter having a frequency characteristic of passing the ultrasonic beam Ba having the frequency Fa but blocking the ultrasonic beam Bb having the frequency Fb that is lower than the frequency Fa and is so configured as to be capable of choosing between passing and not passing the reception signal outputted from the corresponding transducer 3 through the high-pass filter under the control of the reception controller 10.

Next, the operation of Embodiment 1 will be described referring to FIG. 3.

It is assumed that, previously, the measuring conditions stored in the storage unit 23 have been read by the apparatus body controller 21, wirelessly transmitted from the apparatus body controller 21 to the ultrasound probe 1 via the communication controller 20 and the wireless communication unit 14, and inputted to the probe controller 12 via the wireless communication unit 7 and the communication controller 11 of the ultrasound probe 1.

Upon ultrasound diagnosis being started, firstly the probe controller 12 causes the transmission driver 8 to operate via the transmission controller 9, and the transmission driver 8 supplies driving signals to the transducers 3 of all the channels of the transducer array, transmitting the ultrasonic beam Ba for measuring the shallow region A having the frequency Fa and the wave number Ma at time T1. Immediately after the transmission of the ultrasonic beam Ba, the ultrasonic echoes from individual points in the shallow region A are received by the respective transducers 3, which then output respective reception signals, as the probe controller 12 controls the ON/OFF operation of the switches of the channel selector 4 so that the number of channels simultaneously available for reception becomes the number Na=16 previously set for the shallow region A.

Thus, the switches of the channel selector 4 corresponding to 16 channels out of all the 48 channels are turned on while he switches corresponding to the remaining 32 channels are turned off. The 16 channels may be selected, for example, so that they are 16 channels simultaneously available for reception substantially evenly spaced over the whole transducer array or 16 channels centrally located among all the 48 channels of the transducer array.

Ultrasound diagnosis using the ultrasonic beam Ba at time T1 is thus started, and at time T2, when the given time interval ΔT has elapsed after the transmission of the ultrasonic beam Ba, the probe controller 12 again causes the transmission driver 8 to operate via the transmission controller 9, and the transmission driver 8 supplies driving signals to the transducers 3 of all the channels of the transducer array, transmitting the ultrasonic beam Bb for measuring the deep region B. The ultrasonic beam Bb has the frequency Fb that is lower than the frequency Fa of the ultrasonic beam Ba for measuring the shallow region A and the wave number Mb that is different from the wave number Ma of the ultrasonic beam Ba.

Immediately after the transmission of the ultrasonic beam Bb, the ultrasonic echoes from individual points in the shallow region A are received by the respective transducers 3, as the reception controller 10 controls the reception signal processors 5 corresponding to the 16 channels now made simultaneously available for reception to block the ultrasonic beam Bb having the frequency of Pb with the built-in high-pass filters. Therefore, sample data corresponding only to the ultrasonic beam Ba for measuring the shallow region A reflected by and returned from the points in the shallow region A are produced by the reception signal processors 5.

Production of sample data corresponding only to the ultrasonic beam Ba is thus performed, and when ultrasonic echoes corresponding to the ultrasonic beam Ba and traveling from a deepest position in the shallow region A, i.e., the boundary between the shallow region A and the deepest region B, are received at time T3, the reception of the ultrasonic echoes for the shallow region A is terminated.

Thereafter, at time T4, the ultrasonic echoes corresponding to the ultrasonic beam Bb for measuring the deep region B transmitted at a timing delayed by the given time interval AT with respect to the ultrasonic beam Ba for measuring the shallow region A and returning from the most shallow position in the deep region B, i.e., the boundary between the shallow region A and the deepest region B, are received, as the probe controller 12 turns on all the switches of the channel selector 4 so that all the 48 channels of the transducer array become the channels simultaneously available for reception. The reception signal processors 5 of the 48 channels simultaneously available for reception are controlled by the reception controller 10 so as to pass the ultrasonic beam Bb having the frequency Pb without using the built-in high-pass filter. Thus, sample data corresponding to the ultrasonic beam Bb for measuring the deep region B reflected by and returned from the points in the deep region B are produced by the reception signal processors 5.

Production of sample data corresponding to the ultrasonic beam Bb is thus performed, and when ultrasonic echoes corresponding to the ultrasonic beam Bb and returning from the deepest position in the deep region B are received at time T5, the reception of the ultrasonic echoes for the deep region B is terminated, completing one round of ultrasonic wave transmission/reception process for the whole imaging region including the shallow region A and the deep region B.

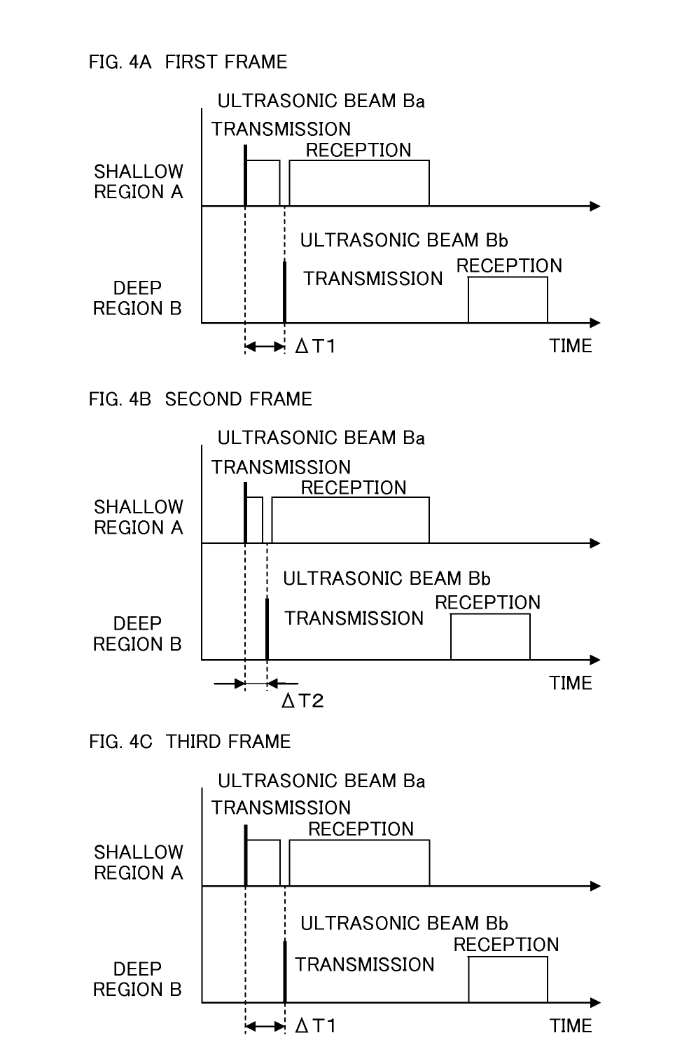

At time T2, when the ultrasonic beam Bb for measuring the deep region B is transmitted, the ultrasonic echoes from the individual points in the shallow region A are received, but the reception of ultrasonic echoes from the shallow region A is impossible during transmission of the ultrasonic beam Bb and is interrupted as illustrated in FIGS. 4A to 4C because transmission and reception are performed by the same transducer array. However, the reception signal for the period during which the reception is interrupted can be produced by performing a frame correlation processing by changing the time interval ΔT in transmission between the ultrasonic beam Ba and the ultrasonic beam Bb in each frame.

The time interval in transmission between the ultrasonic beam Ba and the ultrasonic beam Bb is set, for example, to ΔT1 in the first and the third frame as illustrated in FIGS. 4A and 4C, whereas in the second frame, the time interval in transmission between the ultrasonic beam Ba and the ultrasonic beam Bb is set to a value ΔT2 that is different from the time difference ΔT1 set in the first and the third frame as illustrated in FIG. 4B. Then, an image is produced for a depth for which the reception is interrupted based on the correlation with the preceding and the following frame. For example, a missing portion of the reception signal in the second frame is produced from data for the corresponding depth in the first and the third frame.

In this case, the frame correlation processing is preferably performed for a region sufficiently shallow not to adversely affect the diagnosis, with the time intervals ΔT1 and ΔT2 set to small values.

The sample data thus produced by the respective signal processors 5 are sequentially converted into serial data through the parallel/serial converter 6 before being wirelessly transmitted from the wireless communication unit 7 to the diagnostic apparatus body 2. The sample data received by the wireless communication unit 14 of the diagnostic apparatus body 2 are converted into parallel data through the serial/parallel converter 15 and stored in the data storage unit 16. Further, the sample data are read out from the data storage unit 16 frame by frame, and the image producer 17 generates image signals, based on which image signals the display controller 18 causes the monitor 19 to display an ultrasound diagnostic image.

As is apparent from FIG. 3, the time T3 at which the reception of ultrasonic echoes from the shallow region A ends is different from the time T4 at which the reception of ultrasonic echoes from the deep region B starts by the given time interval LT correspond to the difference in transmission timing between the ultrasonic beam Ba and the ultrasonic beam Bb. Therefore, the image producer 17 performs phasing addition considering the given time interval ΔT using sample data based on the ultrasonic beam Ba for measuring the shallow region A and sample data based on the ultrasonic beam Bb for measuring the deep region B to form a same frame.

As described above, a plurality of ultrasonic beams having different frequencies from each other are sequentially transmitted for the shallow region A and the deep region B, the number Na of channels simultaneously available for reception for the shallow region A is set to a number smaller than the number Nb of channels simultaneously available for reception for the deep region B, and ultrasonic echoes having frequencies each corresponding to the shallow region A and the deep region B are received to form the same frame. Accordingly, the electric power consumed by the reception signal processors 5 is reduced, and the amount of heat generated by the ultrasound probe is also reduced. Thus, temperature rise in the ultrasound probe I can be suppressed while ultrasound diagnosis is continued.

As for the shallow region A, the number Na of channels simultaneously available for reception is set to a number smaller than the number Nb for the deep region B, and the ultrasonic beam Ba having a relatively high frequency Fa is used, so that decrease in image quality is effectively suppressed.

As for the deep region B, not only the number Nb of channels simultaneously available for reception is set to a number larger than the number Na for the shallow region A but the ultrasonic beam Bb having a relatively low frequency Fb is used, so that attenuation occurring as the beam propagates is smaller, enabling a high-quality image to be obtained.

Further, use of different wave numbers Ma and Mb for the ultrasonic beams Ba and Bb makes separation of the ultrasonic beams Ba and Bb easier, enabling high-accuracy measuring.

The frequency Fa of the ultrasonic beam Ba for measuring the shallow region A and the frequency Fb of the ultrasonic beam Bb for measuring the deep region B may be set respectively to, for example, a frequency higher than and a frequency lower than the central frequency in the frequency band used by the ultrasound probe 1.

### Embodiment 2

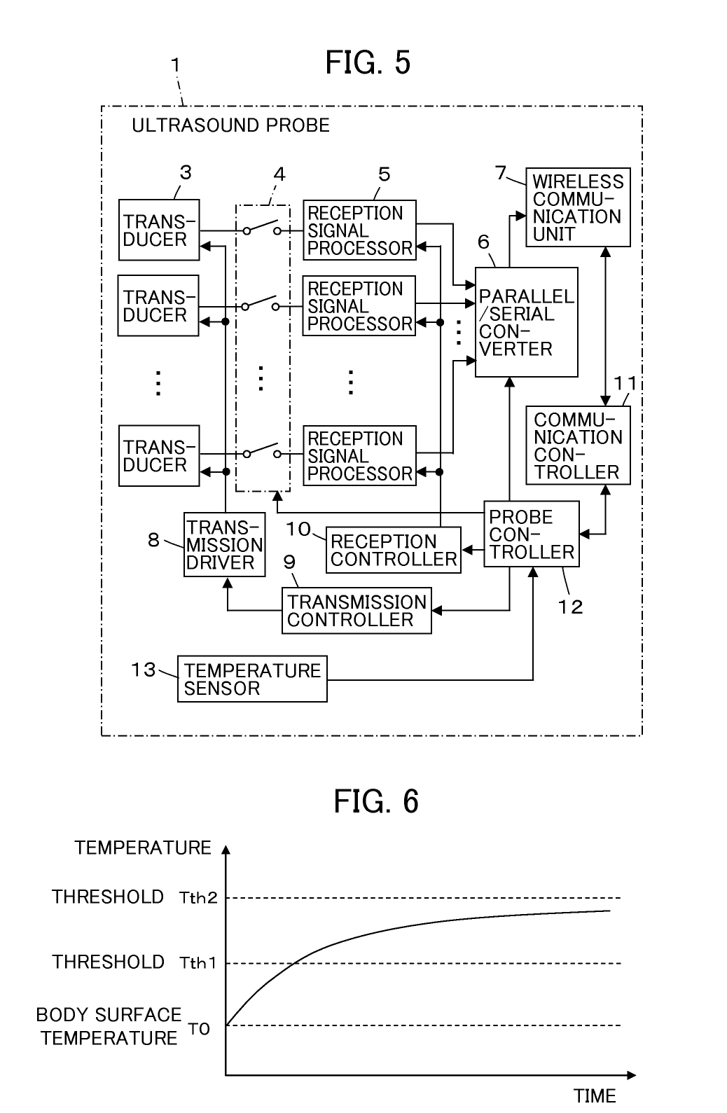

FIG. 5 illustrates a configuration of an ultrasound probe 31 used in an ultrasound diagnostic apparatus according to Embodiment 2. The ultrasound probe 31 has the same components as the ultrasound probe 1 used in Embodiment 1 illustrated in FIG. 1 except that the ultrasound probe 31 includes a temperature sensor 13 connected to the probe controller 12.

The temperature sensor 13 detects an internal temperature T the ultrasound probe 31 and outputs the detected internal temperature T to the probe controller 12.

As illustrated in FIG. 6, a first temperature threshold Tth1 higher than a subject's body surface temperature T0 (about 33° C.) is previously set, and a second temperature threshold Tth2 higher than the first temperature threshold Tth1 is also previously set.

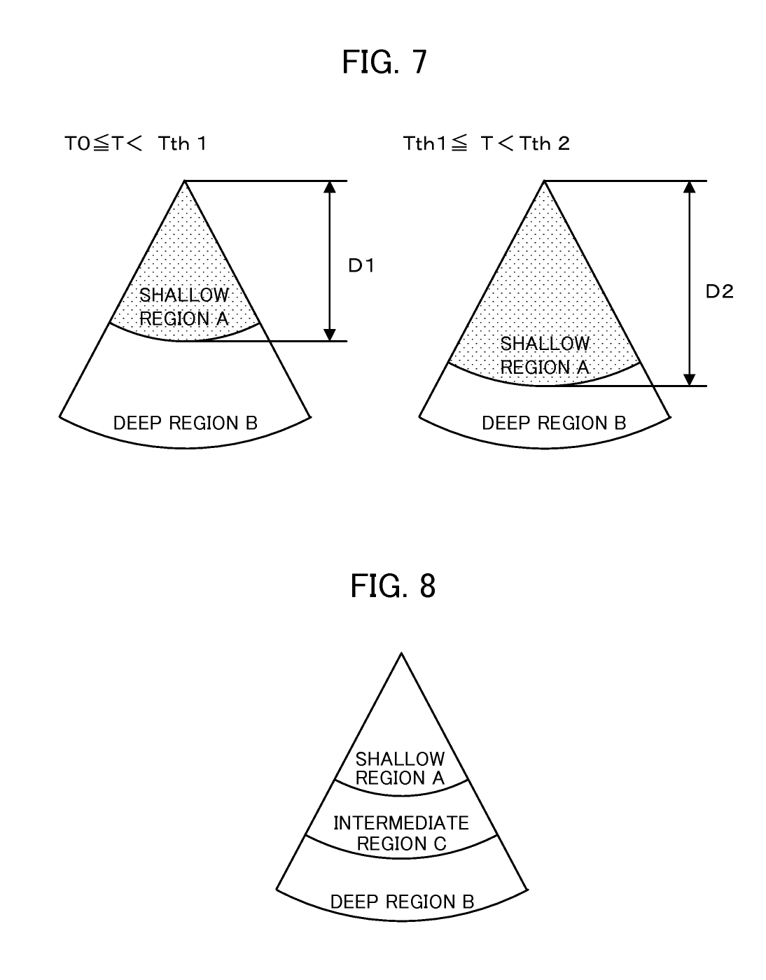

As illustrated in FIG. 7, when the internal temperature of the ultrasound probe 31 is T0≦T<Tth1, the shallow region A and the deep region B are divided at a first depth D1, while when Tth1 ≦T<Tth2, the shallow region A and the deep region B are divided at a second depth D2 that is deeper than the first depth D1.

Thus, according to Embodiment 2, when the internal temperature T of the ultrasound probe 31 reaches or exceeds the first temperature threshold Tth1, the shallow region A, for which the number of channels simultaneously available for reception is reduced, is expanded.

The first temperature threshold Tth1 and the second temperature threshold Tth2 may be set respectively to, for example, 37° C. and 43° C., and stored with the first depth D1 and the second depth D2 in the storage unit 23 of the diagnostic apparatus body 2.

When ultrasound diagnosis is started, the internal temperature T of the ultrasound probe 31 is first detected by the temperature sensor 13 and wirelessly transmitted to the diagnostic apparatus body 2 via the probe controller 12, the communication controller 11, and the wireless communication unit 7. The internal temperature T received by the wireless communication unit 14 of the diagnostic apparatus body 2 is inputted to the apparatus body controller 21 via the communication controller 20.

The apparatus body controller 21 reads the first temperature threshold Tth1 and the second temperature threshold Tth2 stored in the storage unit 23 and compares the entered internal temperature T of the ultrasound probe 31 with the first temperature threshold Tth1 and the second temperature threshold Tth2. One of the first depth D1 and the second depth D2 is selected by the apparatus body controller 21 depending on the comparison result and wirelessly transmitted together with the other measuring conditions previously stored in the storage unit 23 to the ultrasound probe 31 via communication controller 20 and the wireless communication unit 14 and inputted to the probe controller 12 via the wireless communication unit 7 and the communication controller 11 of the ultrasound probe 31.

Subsequently, transmission and reception of the ultrasonic waves for the shallow region A and the deep region B are performed likewise as in Embodiment 1, and the ultrasound diagnostic image produced by the image producer 17 of the diagnostic apparatus body 2 is displayed on the monitor 19.

Thus, consumption of electric power and generation of heat can be further reduced by expanding the shallow region, for which the number of channels simultaneously available for reception is reduced, when the internal temperature T of the ultrasound probe 31 reaches or exceeds the first temperature threshold Tth1.

When the internal temperature T of the ultrasound probe 31 increases to a temperature equal to or above the second temperature threshold Tth2, transmission and reception of the ultrasonic waves are terminated until the internal temperature T decreases again to under the second temperature threshold Tth2.

The temperature sensor 13 is preferably located near the reception signal processors 5, where heat is expected to develop during the operation of the ultrasound diagnostic apparatus.

Although two temperature ranges, T0≦T<Tth1 and Tth1≦T<Tth2, are used to judge the internal temperature T of the ultrasound probe 31, other temperature ranges, say three or more temperature ranges, for example, may be used to judge the internal temperature T of the ultrasound probe 31. In this case, the shallow region A is expanded increasingly as the internal temperature T of the ultrasound probe 31 rises.

### Embodiment 3

While, in Embodiments 1 and 2, the imaging region is divided into two regions, the shallow region A and the deep region B according to the measuring depth, the invention is not limited this way. As illustrated in FIG. 8, for example, with imaging region divided into three region, the shallow region A, the intermediate region C, and the deep region B according to the measuring depth, three ultrasonic beams having different frequencies and transmission timings from each other may be transmitted for these regions, and ultrasonic echoes may be received with different numbers of channels simultaneously available for reception.

In this case, preferably a same frame is formed by increasingly reducing the number of channels simultaneously available for reception while transmitting an ultrasonic beam having an increasingly higher frequency as the measuring depth for a region decreases of the shallow region A, the intermediate region C and the deep region B, and by receiving ultrasonic echoes having a frequency corresponding to each of the regions.

Likewise, the imaging region may be further divided into four or more regions according to the measuring depth.

In Embodiment 3 also, as in Embodiment 2, the internal temperature T of the ultrasound probe may be detected, so that the shallow region A and the intermediate region C, for which the number of channels simultaneously available for reception is reduced, may be increasingly expanded as the detected internal temperature T rises.

While the measuring conditions are stored in the storage unit 23 of the diagnostic apparatus body 2 in Embodiments 1 and 2, the measuring conditions may alternatively be stored in the ultrasound probe 1 to sequentially transmit a plurality of ultrasonic beams having different frequencies corresponding to a plurality of measuring depth regions, increasingly reduce the number of channels simultaneously available for reception for the measuring depth regions as the measuring depth decreases, and receive ultrasonic echoes each having a frequency corresponding to each of the measuring depth regions to form the same frame.

While the ultrasound probe 1 or 31 described in Embodiments 1 to 3 comprises a transducer array having a total of 48 channels by way of example, the number of channels, 48, is only illustrative, and the present invention may likewise be applied to an ultrasound probe comprising a transducer array having another number of channels.

Although the ultrasound probe 1 or 31 and the diagnostic apparatus body 2 are connected to each other by wireless communication in Embodiments 1 to 3, the invention is not limited to such configuration; the ultrasound probe 1 or 31 may be connected to the diagnostic apparatus body 2 via a connection cable. Such configuration obviates the necessity of providing the wireless communication unit 7 and the communication controller 11 of the ultrasound probe 1 or 31, the wireless communication unit 14 and the communication controller 20 of the diagnostic apparatus body 2, and the like.

What is claimed is:

1. An ultrasound diagnostic apparatus comprising:
an ultrasound probe including a transducer array with a plurality of channels;
a transmission driver for transmitting an ultrasonic beam from the transducer array toward a subject;
reception signal processors for processing reception signals outputted from the transducer array having received ultrasonic echoes from the subject;
an image producer for producing an ultrasound image based on the reception signals processed by the reception signal processors;
a channel selector for selecting channels that are simultaneously available for reception from the plurality of channels; and
a controller for controlling the transmission driver to sequentially transmit a plurality of ultrasonic beams having different frequencies corresponding to a plurality of measuring depth regions from the transducer array, controlling the reception signal processors and the image producer to form a same frame by receiving ultrasonic echoes having a frequency corresponding to each of the plurality of measuring depth regions, and controlling the channel selector to increasingly reduce the number of channels simultaneously available for reception as a measuring depth in the plurality of measuring depth regions decreases.

2. The ultrasound diagnostic apparatus according to claim 1, wherein the controller controls the transmission driver to transmit an ultrasonic beam having an increasingly higher frequency as the measuring depth in plurality of measuring depth regions decreases.

3. The ultrasound diagnostic apparatus according to claim 1, further comprising a temperature sensor for detecting an internal temperature of the ultrasound probe, the controller increasingly expanding a region for which the number of channels simultaneously available for reception is reduced as an internal temperature detected by the temperature sensor increases.

4. The ultrasound diagnostic apparatus according to claim 2,
further comprising a temperature sensor for detecting an internal temperature of the ultrasound probe,
the controller increasingly expanding a region for which the number of channels simultaneously available for reception is reduced as an internal temperature detected by the temperature sensor increases.

5. The ultrasound diagnostic apparatus according to claim 1, wherein the controller controls the transmission driver and the reception signal processors to transmit and receive ultrasonic beams having different wave numbers corresponding to the plurality of measuring depth regions.

6. The ultrasound diagnostic apparatus according to claim 2, wherein the controller controls the transmission driver and the reception signal processors to transmit and receive ultrasonic beams having different wave numbers corresponding to the plurality of measuring depth regions.

7. The ultrasound diagnostic apparatus according to claim 3, wherein the controller controls the transmission driver and the reception signal processors to transmit and receive ultrasonic beams having different wave numbers corresponding to the plurality of measuring depth regions.

8. The ultrasound diagnostic apparatus according to claim 4, wherein the controller controls the transmission driver and the reception signal processors to transmit and receive ultrasonic beams having different wave numbers corresponding to the plurality of measuring depth regions.

9. An ultrasound image producing method, comprising the steps of:
sequentially transmitting a plurality of ultrasonic beams having different frequencies corresponding to a plurality of measuring depth regions from a transducer array in an ultrasound probe;
increasingly reducing the number of channels simultaneously available for reception as a measuring depth in the plurality of measuring depth regions decreases; and
forming a same frame by receiving ultrasonic echoes having a frequency corresponding to each of the plurality of measuring depth regions to produce an ultrasound image.

10. The ultrasound image producing method according to claim 9, wherein an ultrasonic beam having an increasingly higher frequency is transmitted as the measuring depth in the plurality of measuring depth regions decreases.

11. The ultrasound image producing method according to claim 9,
wherein an internal temperature of the ultrasound probe is detected, and
wherein a region for which the number of channels simultaneously available for reception is reduced is increasingly expanded as a detected internal temperature of the ultrasound probe increases.

12. The ultrasound image producing method according to claim 10,
wherein an internal temperature of the ultrasound probe is detected, and
wherein a region for which the number of channels simultaneously available for reception is reduced is increasingly expanded as a detected internal temperature of the ultrasound probe increases.

13. The ultrasound image producing method according to claim 9, wherein ultrasonic beams having different wave numbers corresponding to the plurality of measuring depth regions are transmitted and received.

14. The ultrasound image producing method according to claim 10, wherein ultrasonic beams having different wave numbers corresponding to the plurality of measuring depth regions are transmitted and received.

15. The ultrasound image producing method according to claim 11, wherein ultrasonic beams having different wave numbers corresponding to the plurality of measuring depth regions are transmitted and received.

16. The ultrasound image producing method according to claim 12, wherein ultrasonic beams having different wave numbers corresponding to the plurality of measuring depth regions are transmitted and received.
